(19) United States
(12) Patent Application Publication
(10) Pub. No.: US 2007/0092958 A1
(43) Pub. Date: Apr. 26, 2007

(54) METHOD AND APPARATUS FOR GUIDING GROWTH OF NEURONS

(76) Inventors: Naweed I. Syed, Calgary, AB (CA); Graham Arnold Jullien, Calgary, AB (CA)

Correspondence Address: FISH & RICHARDSON P.C., PO BOX 1022, MINNEAPOLIS, MN, 55440-1022, US

(21) Appl. No.: 11/458,088

(22) Filed: Jul. 17, 2006

Related U.S. Application Data
(60) Provisional application No. 60/699,829, filed on Jul. 15, 2005.

(51) Int. Cl. C12N 13/00; C12M 1/36

(52) U.S. Cl. 435/173.8; 435/305.1; 435/286.1

(57) ABSTRACT

This invention pertains to a method and apparatus for facilitating guided growth of axons and dendrites in cell culture, for example for studies of axonal pathfinding, target cell selection, synapse formation, synaptic physiology, neuronal plasticity, drugs screening and gene perturbations. In a preferred embodiment, the invention includes a semiconducting substrate surface containing an array of capacitors that directly stimulate and read from neurons cultured on the surface. The chip may also have patterns of growth permissive substances, including Schwann cells, and/or trophic molecules that enable rapid and directed growth of axons/dendrites from cultured neurons.

### CLAIM OF PRIORITY

This application claims the benefit of priority under 35 U.S.C. § 119 to U.S. provisional application Ser. No. 60/699,829, filed Jul. 15, 2005, the disclosure of which is incorporated herein by reference in its entirety.

### RELATED APPLICATIONS

The disclosures of U.S. utility applications having Ser. No. 11/439,377 filed May 22, 2006, Ser. No., 11/423,380 filed Jun. 9, 2006, Ser. No. 11/455,222 filed Jun. 15, 2006, and Ser. No. 11/424,413 filed Jun. 15, 2006, are also incorporated herein by reference in their entirety.

### FIELD OF THE INVENTION

The present invention pertains generally to the guided growth of axons and dendrites in cell culture for control of axonal pathfinding, target cell selection, synapse formation, synaptic physiology, neuronal plasticity, drug screening, and carrying out gene perturbations and monitoring their effects. Specifically, the invention pertains to a silicon chip device that allows the generation of arbitrarily selected electric fields for controlled stimulation of neurons cultured on the chip.

### BACKGROUND

Interfaces between the electrical connections of the nervous system that evolved over aeons in the natural world, and the silicon-based microcircuitry that man has developed in only the last half-century, are the core of a new generation of revolutionary technologies (see, e.g., K Boahen, “Neuromorphic Microchips”, Scientific American, vol. 292, no 5, pp 56-63, May 2005). In contrast to approaches that electronically emulate neurons or that join neuronal networks with electronics by impaled microelectrodes, a direct non-invasive interfacing of semiconductor chips and nerve cells can lead to real hybrids that are microscopically integrated.

The nervous system is composed of neurons and glial cells, which include, for example, astroglia, microglia, and Schwann cells. The neurons respond to stimuli and carry signals between the brain and the rest of the body, while the glial cells provide support for the neurons and influence the speed of electrical signals. Each neuron has a cell body and neurite processes, including an axon and dendrites, extending out from the cell body towards other neurons or to other types of cells such as muscle cells. The tip of an axon is a growth cone and is responsible for navigation of a growing axon in the direction of a neighboring neuron. Unlike most other types of cells, neurons do not grow by dividing to form colonies but, rather, the membrane of a neuron grows (extends) to form axons/dendrites. Neuronal growth therefore generally refers to growth of axons and dendrites. Collections of neurons are referred to as bundles, or as neuronal networks. A single neuron can make multiple contacts, called synapses, with other neurons through its axon and its dendrites. Impulses are transmitted through the synapses from a first neuron, the pre-synaptic neuron, to a second neuron, the post-synaptic neuron. The synaptic connectivity between any pair of neurons is not hard-wired but rather exhibits a high degree of plasticity. Information processing in the central nervous systems (CNS) is primarily mediated through synaptic connections, which remain modifiable throughout life. These connections determine how large networks of neurons may coordinate their ensemble output to effect such complex functions of the CNS, as learning and memory formation.

Much of what is known about nerve growth has been discovered by studying damaged neurons and their attendant repair mechanisms. When a nerve is severed, a gap is formed between the separated segments of the injured nerve. The nerve axon must navigate and bridge the gap in order to regenerate and re-establish nerve function. Under appropriate conditions, the end that is proximal to the cell body forms a new growth cone that navigates the gap. The growth cone and the dendrites on the proximal end typically grow in many directions and, unless the injury is small, the growth cone may never reach the distal segment. Axon injury also stimulates production of trophic factors such as nerve growth factors, which greatly enhance the growth of neurons in culture. Regenerating axons stimulate Schwann cells to proliferate and form a basal lamina of collagen, proteoglycans, and laminin. The direction of growth and movement of the axon is also responsive to environmental signals provided by other cells.

Cultured neurons—such as in cell or organ culture—are a primary tool for investigating both the molecular and cellular mechanisms that underly neurite outgrowth, cell migration, target cell recognition, synaptic connectivity, synapse formation, aspects of synaptic physiology such as synaptic plasticity, and the complex functions of the nervous system, as well as nerve regeneration. Cultured neurons are also used for drug testing and the study of gene perturbations. However, there are a number of problems associated with their use.

First, the neurite extension process in cell cultures is often stochastic, and it is only through chance that any two cultured neurons may establish the physical contacts necessary to develop synapses. This undirected search for target cells by potential partners often takes weeks, rendering the neurons devoid of the protein pool that is essential for viable, long-term cultures. Second, monitoring and studying the activities of large, random neuronal networks simultaneously and non-invasively at the cellular and molecular levels is technically challenging. Moreover, it is difficult, if not impossible, to selectively target individual neurons in cultured networks for fluorescent labeling and gene targeting. Often, expensive and laborious means of intracellular labeling or gene perturbations are used to target the cells of interest. Such processes have a very low success rate. Consequently, at present, no reliable tools are available to culture neuronal cells under precisely controlled conditions in order to study the mechanisms by which growth cones of developing or injured neurons find their path en route towards their targets and how this growth is affected by extrinsic factors. This information is important for designing strategies that would be required for successful regeneration after nerve injuries in humans.

Several techniques have been reported that attempt to aid and control neuronal growth by culturing neurons on a substrate, but they have been successful only to a limited extent. For example, fabricated grooves on substrate surfaces have been employed to guide neuronal growth (Clark, et al., “Topographical control of cell behaviour: II. Multiple grooved substrata.” Development, (1990), vol. 108, 635-644, incorporated herein by reference). However, alignment of neurons using substratum topographical cues alone is highly uncertain and difficult to reproduce. James, et al., (IEEE Transactions on Biomedical Engineering, (2000), vol. 47, 17-21, incorporated herein by reference), used microcontact printing to produce poly-L-lysine micropattems on silicon substrates. Cultured neurons selectively adhered to poly-L-lysine lines and grew neurites along the lines. Kam, et al., (“Axonal outgrowth of hippocampal neurons on micro-scale networks of polylysine-conjugated laminin.” Biomaterial, (2001), vol. 22, 1049-1054, incorporated herein by reference) also showed that micropatterns of poly-lysine-conjugated laminin enabled neurons to adhere and extend axonal processes along the prescribed patterns. In these studies, the chemical stimuli are preprinted on the substrate uniformly according to the micropatterns. A drawback of preprinted substrates is that the substrates cannot provide effective guidance cues right around or near the growth cone of the neuron, as the brain provides to developing or injured neurons. Furthermore, the direction in which the neurites are to grow is pre-determined by the chemical patterns on the substrates. Since the chemical patterns attract all nearby neurons, multiple neurons can also grow along the same lines. It would be difficult to use these substrates to precisely guide the growth of just two neurons in real time to promote synapse formation and to study synapse function.

In addition to spatial and chemical cues, electrical signals can provide additional stimulation cues for neuronal growth. Recently, neuronal cell networks have been constructed on planar microelectrode arrays consisting of transistors. See, e.g., James, et al., (“Extracellular Recordings from Patterned Neuronal Networks Using Planar Microelectrode Arrays” IEEE Transactions on Biomedical Engineering, (2004), vol. 51, 1640-1648, incorporated herein by reference). The microelectrode array was used for extracellular recording of firing activities of the neurons, rather than for stimulating the cells. Kaul, et al. (“Neuron-Semiconductor Chip with Chemical Synapse between Identified Neurons,” Physical Review Letters, (2004), vol. 92(3), 038102, incorporated herein by reference) developed a hybrid device by incorporating both a transistor and a capacitor, instead of a simple microelectrode, at each node of a linear silicon array. Individually identifiable pre- and post-synaptic neurons were directly cultured on the silicon chip in a soma-soma (cell body) configuration. The capacitor induced synaptic potentiation in the presynaptic neuron and the transistor recorded the action potentials in the juxtaposed presynaptic and postsynaptic neurons. This study demonstrated that a capacitor can be used to directly modulate synapses between two neurons on a silicon chip. Additionally, Keilman, et al., (IEEE International Workshop on Biomedical Circuits and Systems, (2004), incorporated herein by reference) described a lexel (electric field element) array microchip composed of a programmable, two-dimensional array of independent microelectrodes that creates a non-uniform electric field. The electric field shapes were programmed to move charged microscopic particles around. However, none of these reports address how the growth of individual neurons can be controlled.

Molecular and cellular techniques like living cell imaging and gene perturbation are important tools for studying how various factors modulate neurite growth and synapse formation. Yet no convenient and cost-effective means are available to introduce the factors or gene perturbation molecules into individual neurons in cell culture.

Recently, conjugates of quantum dots (QD's) have become available as fluorescent probes in biological applications (see, e.g., Bruchez Jr., M., et al., Science, 281, 2013-2016 (1998), and Chan, W. C., and Nie, S., Science, 281, 2016-2018, (1998), both of which are incorporated herein by reference). QD's are nanoparticles whose surface fabrication enables selective binding to biomolecules, such as antibodies and proteins, and also to trophic factors. Although the absorption spectra of QD's are very broad, their emission is characteristically confined to a narrow band, which is dependent on the size of the nanoparticles. In comparison with traditional fluorescent dyes such as rhodamine, QD's possess unique optical properties that are advantageous for biological imaging, such as extended photostability, multicolor excitation, and high brilliance that permits detection of a single nanoparticle. Since QD's are comparable to the size of proteins (5-15 nm) and have controllable surface properties, more recently they have been proposed as a tool to deliver biomolecules and other exogenous drug compounds to cellular targets. Vu, et al. (“Peptide-Conjugated Quantum Dots Activate Neuronal Receptors and Initiate Downstream Signaling of Neurite Growth”, Nano letters, (2005), vol. 5, 603-607, incorporated herein by reference) showed that the surfaces of commercial QD's conjugated with the beta subunit portion of NGF (βNGF) retained the bioactivity of βNGF. These QD-βNGF conjugates effectively activated TrkA receptors (trophic factor receptors), and evoked downstream cellular signaling cascades to initiate neuronal differentiation in PC12 cells (neuronal tumor cell line). Additionally, Gur and Yarden described how various trophic factors such as Epidermral Growth Factor (EGF) can be coated onto quantum dots/nano beads to activate biological processes (G. Gur and Y. Yarden, “News and Views,” Nature Biotechnology, (2004), Vol. 22, pages 169-170, incorporated herein by reference).

However, precise control of the time and the site of delivery of the QD conjugates to individual neurons is a challenge that needs to be solved. This, coupled with the fact that neuronal growth patterns in cell culture are uncontrolled and often random, renders data collection difficult.

Because cell or organ culture techniques are, at present, the only effective means used to define the role of various intrinsic and extrinsic factors in regulating the cellular and molecular mechanisms of nerve regeneration, synapse formation, synaptic physiology and plasticity, further progress in this field is significantly hampered for the reasons discussed hereinabove. There therefore exists a need in the art for a device and method for guiding the growth of axons/dendrites in a highly controlled manner and is also capable of delivering species such as fluorescent markers and gene manipulation molecules to selected neurons.

The discussion of the background to the invention herein is included to explain the context of the invention. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY OF THE INVENTION

The present invention pertains generally to the guided growth of axons and dendrites in cell culture. Specifically, the present invention relates to methods and devices designed to strategically guide the growth of cultured neurons along electric fields produced by a well-defined array of electrodes coupled with growth-permissive substances such as Schwann cells, and optionally a patterned deposition of trophic factors on a substrate or well structure. More specifically, the invention pertains to a silicon chip device containing an array of electrodes that allow the generation of arbitrary electric fields above the chip, and also directly allow the stimulation of neurons cultured on the chip. An array of electric field sensors may also be included for monitoring neuron electrical activity.

The present invention includes a process for guiding growth of neurons, the process comprising: culturing neurons on a substrate surface; patterning growth permissive substances, trophic factors, and nano particles on the substrate surface; fabricating an array of tiles on the surface; and applying an electrical voltage to at least one of the tiles, thereby stimulating growth in a neuron in contact with the at least one tile.

The present invention includes a system for guiding growth of neurons, the system comprising: an array of tiles on a surface, wherein each tile in the array of tiles is preferably independently electrically addressable; a medium contacting the surface, wherein the medium contains: one or more neurons, and growth permissive substances; and a microcontroller electrically connected to the array of tiles, and configured to cause an electrical voltage to be applied selectively to at least one of the tiles, thereby causing one or more neurons to grow.

A number of mechanisms for stimulating guided neuronal growth are consistent with the methods and apparatus of the present invention. In a first embodiment, growth is stimulated by the application of a field alone. In this embodiment, the neurons are cultured on a surface having an array of tiles, in the presence of growth permissive substances. The application of voltages at selectable tiles causes spatially varying fields, i.e., a field gradient, on the surface to promote neuronal growth in specific directions. In a further embodiment, the growth permissive substances can be patterned on the surface, to give further stimulus to the directional growth of the neurons. In a still further embodiment, growth is stimulated by a combination of application of an electric field and growth enhancing molecules, such as trophic factors. The trophic factors can be patterned on the substrate, for example, by microprinting, or can be delivered to specified locations by micropumping. Alternatively, the trophic factors can be bound to nanoparticles, which themselves can be arranged in a pattern on the surface. The nanoparticles can be caused to liberate the trophic factors by selective application of an electric field. The pattern of the trophic factors and/or nanoparticles can come from positioning of the substances in a well associated with each tile.

### BRIEF DESCRIPTION OF THE DRAWINGS

FIG. 1 shows a flow-chart of an overall process according to the present invention;

FIG. 2 shows an apparatus for performing a process of the present invention;

FIG. 3 shows a circuit for controlling the apparatus of FIG. 2;

FIG. 4 shows a silicon chip interfaced with a synapse;

FIG. 5 shows neurons with soma-soma synapse on a silicon chip;

FIG. 6 shows synaptic transmission on silicon chip; and

FIG. 7 illustrates synaptic potentiation on a silicon chip.

### DETAILED DESCRIPTION

Overview

The present invention relates to systems and methods that promote guided growth of axons and dendrites along the surface of a substrate such as a silicon die. In particular, the systems and methods facilitate growth rates of neurons that are more rapid than previously known in the art. The devices provided herein can provide electric fields (e.g., by application of electrical pulses), chemical agents (e.g., chemical, trophic, and substrate adhesion molecules), or a combination of both, at selected areas of a substrate to promote nerve growth. In general, neurons grow along a gradient from a lower concentration of trophic factors to higher concentrations.

Specifically, this invention pertains to the guided growth of axons and dendrites along time-varying electric fields generated from arrays of capacitors. The electric fields can be varied based on, for example, the monitoring of growth cone progress along specified directions, and the use of electric field waveforms that have been experimentally shown to enhance growth. The invention allows for the application of temporal and spatial variations of electric fields that can be determined from experimental studies.

In particular, the invention provides a substrate containing arrays of electrodes connected to electronic switches that are able to place arbitrary voltages on the electrodes, thus generating arbitrary non-uniform and temporally varying, electric fields above the substrate. In one embodiment, the substrate includes patterns of growth permissive substances and trophic molecules that enable rapid and guided growth of neurons cultured on the surface of the substrate. The patterns can be produced by dielectrophoretic and electrophoretic forces on the molecules (when, respectively, uncharged and charged), induced by the generation of electric fields using the electrode array. The patterns can also be created using microcontact printing.

In one embodiment, the electrodes are also plates of capacitors so that electric fields can be generated between the capacitor plates when a voltage is applied. The capacitors may also be formed around “wells”, etched into the surface of the substrate, which contain the growth permissive substances and trophic molecules. Nanoparticles, such as nano-beads and quantum dots, which will be used to provide growth enhancement and/or as markers, can either be immobilized at specific sites on the surface of the substrate or in the wells, or delivered to the neuronal milieu through, their ‘un-caging’ via electric fields developed across the plates of the capacitors. These nano-beads and quantum dots may be pre-labeled with various markers (such as fluorescent markers, or biological markers) for highly selective neuronal labeling, or may be designed to deliver various protein molecules and gene perturbation molecules. Alternatively, instead of fluorescent nanoparticles, or nanoparticles labeled with fluorescent markers, nano-particles that are visible because of their light-scattering properties can be used. (See, e.g., U.S. Patent Application Publication No. 2002/0028519, to Yguerabide, et al., incorporated herein by reference.)

In certain embodiments, a device provided herein can have an array of transistors and a processor in an integrated circuit form (e.g., a transistor chip) associated with the electrodes.

Method

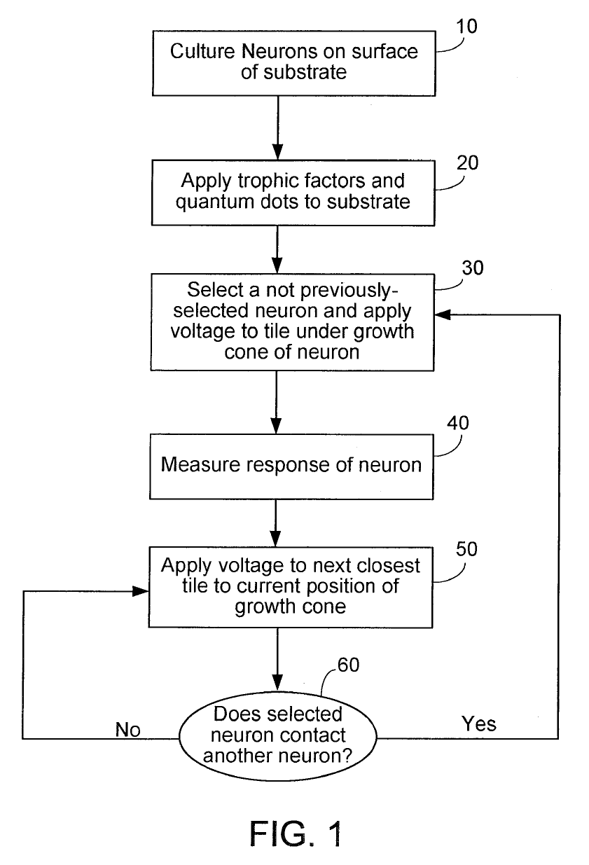

In a method according to the present invention, shown in FIG. 1, neurons are cultured on the surface of substrate, step 10. It would be understood that the present invention could be performed with a substrate that had been predisposed with a neuronal culture. Growth enhancing molecules such as trophic factors, and labeled nano particles such as quantum dots are applied to the substrate, step 20. In certain embodiments, step 20 may precede step 10. Next, a not previously-selected neuron is selected, and a voltage is applied to the tile that is situated under the growth cone of neuron, step 30. The presence of a neuron above a tile can be determined by measuring such quantities as the impedance above the electrode, or a change in resistance (such as obtained by measuring a change in thermal noise). Also used to determine if neuron is present The response of the neuron is measured, step 40, and a voltage is applied to the next closest tile to current position of growth cone, step 50. For example, if the growth cone of the neuron has not appreciably moved since step 30, the voltage is applied to the same tile as was addressed in most recently carried out step 30. Next, the selected neuron is analyzed to see whether it contacts (i.e., forms a synapse with) another neuron, step 60. Presence of a synapse can be determined, e.g., by imaging with a CMOS image sensor+light source. Also, a synapse may be seen by using techniques applied to determining the presence of a neuron, such as measuring the impedance above the electrode, or measuring a change in resistance (thermal noise). If it does not, then a further voltage is applied to the next closest tile to the current position of the growth cone of the selected neuron, step 50. If the first selected neuron does not contact another neuron, then the procedure returns to step 30, and another neuron, not previously selected, is selected.

Apparatus

In one aspect, the invention provides a system that ensures the rapid and guided growth of axons and dendrites, the system comprising: an array of tiles on a surface, wherein each tile in the array of tiles is independently electrically addressable; a medium contacting the surface, wherein the medium contains one or more neurons, growth permissive substances, trophic factors, and nanoparticles, wherein the trophic factors and nanoparticles are independently arranged in a pattern on the surface; and a microcontroller electrically connected to the array of tiles, and configured to cause an electrical voltage to be applied selectively to at least one of the tiles, thereby causing one or more neurons in contact with the at least one tile to grow.

The tile, as used with the present invention, is a region of a semiconductor surface, having electrical connectivity to a microcontroller, and having at least one of switching and sensing functions so that it can cause an electric field to be applied to it surface, and/or can detect the presence of one or more neurons thereon.

The surface on which the neurons are cultured is preferably the surface of a semiconducting die on which is fabricated integrated circuitry that defines an array of a desired number of tiles that further comprise transistors, electrodes, and controllable circuitry. The transistors can be used as switches, for example to switch voltages onto a tile, as well as sensors, for example for sensing an electric field adjacent the tile. Accordingly, the transistors are preferably MOS/MOSFET transistors, which are field-based and so lend themselves to applications as sensors in this manner. MOSFETs are also ideal switches because when the gate is closed, they resemble a resistor. Less preferred are bipolar transistors. The surface is preferably the surface of a chip that comprises a silicon material, and is preferably non-toxic to neurons. The substrate is coated with growth permissive substances such as substrate adhesion molecules, for example, fibronectin, laminin and collagen. These may also be patterned on the surface. Also included on the substrate may be trophic factors, Schwann cells and/or quantum dots, also patterned respectively.

Preferred examples of neurons For use with the present invention include: hippocampal, peripheral neurons such as dorsal root ganglia, cerebral neurons, e.g., purkinje cells.

The surface resembles a lexel array (see, e.g., Keilman, et al., IEEE International Workshop on Biomedical Circuits and Systems, (2004)) but having a pair of electrodes at each element of the array. The electrode structure of the device of the present invention provides charge transfer stimulus to the neurons using the inherent capacitance of each electrode and its upper dielectric layer, thereby allowing the substrate to communicate with individual neurons in the neuronal culture.

Because neuron cells are poorly adhesive for a silicon surface, the surface is patterned with a variety of growth enhancing molecules. For example, the a silicon wafer can be placed in a dish, for this purpose. Specifically, higher concentrations of trophic factors such as nerve growth factor (NGF) and epidermal growth factor (EGF), present in appropriate biological media, such as a saline solution, the compositions of which are understood by one of ordinary skill in the art, in contact with the poly-L-lysine coated silicon wafers in the bottom of a dish for 4-6 hrs prior to the neuronal culture. This permits a uniform deposition and adhesion of trophic factors onto the poly-L lysine surface. The solution containing the trophic factors is then replaced with normal culture medium while the trophic factors remain bound to the surface. Any 2-dimensional pattern of, e.g., growth-enhancing molecules, can be defined by releasing molecules from arbitrarily selected tile wells.

A micro-pump can be configured to deliver one or more growth enhancing molecules selectively underneath each capacitor in a controlled manner. The perfusion time “on/off” mechanisms and the rate of flow can be synchronized and controlled through the same mechanism that regulates capacitor function. The release of the trophic molecules can be through a MEMS micropump with outlet gates on the chip being controlled by the processor so that fluid containing the molecules can be directed to a variety of positions on the substrate.

The substrate surface is also patterned with nanoparticles such as quantum dots (QD's), in a similar manner as the growth enhancing molecules are patterned. Specifically, quantum dots coated with various trophic factors or gene perturbation molecules can either be delivered to specific capacitor sites through a micro-pump and released as per capacitor stimulation mechanisms (synchronous release with the applied electrical field). Alternatively, trophic factor conjugated quantum dots, charged either negatively or positively, and dissolved in biological saline solutions, are added to the surface prior to neuronal culture. A capacitive current of the opposite polarity (to that of the QD) is applied to attract the quantum dots to all or select capacitor sites. The remainder solution is then washed away and dishes are filled with culture medium. To release the QD's the polarity of the capacitive current is reversed (to repel the QD away from the molecule trapping well onto the substrate).

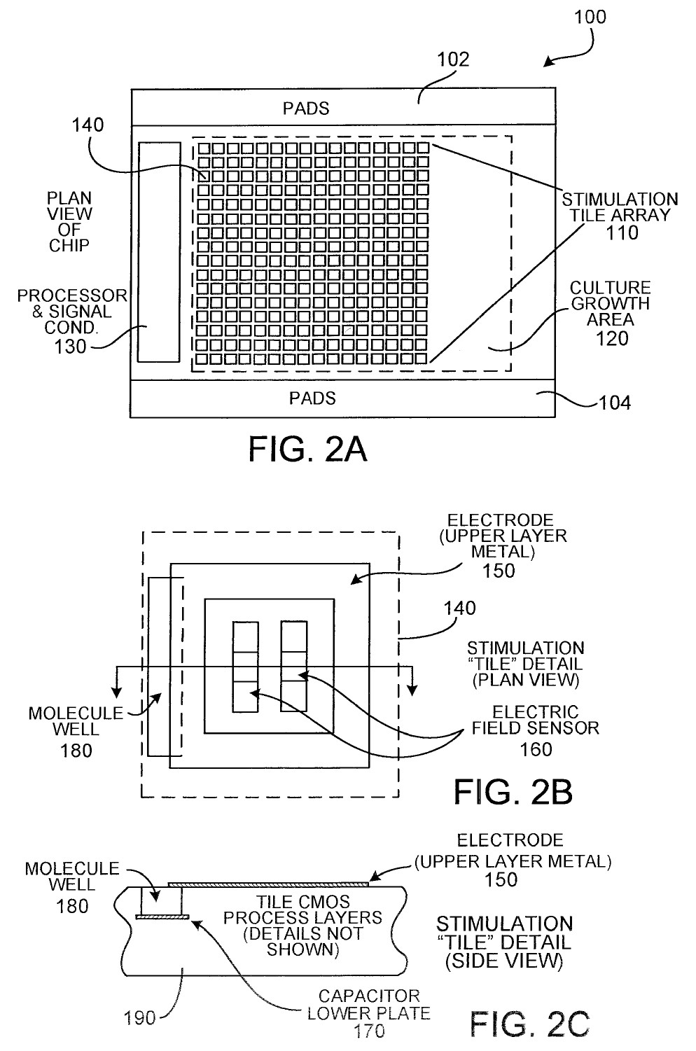

FIG. 2A shows an embodiment 100 of the apparatus of the invention. FIGS. 2B and 2C show a plan and side view, respectively, of a tile in the apparatus of FIG. 2A. In particular, FIGS. 2B and 2C show an embodiment of a tile that includes integration of: an electrode array; molecule trapping wells; capacitive (electric field or charge) release of active molecule factors; and electric field sensors. FIG. 2C shows various layers of a tile. As further described herein, such layers may be fabricated by methods of semiconductor fabrication known in the art.

Ground shields, which are fabricated from conducting material situated between conductors and used to block the interference effect of time varying electric fields between them, allow arbitrary voltages to be switched onto the tile electrodes, thus creating defined electric fields above the substrate surface, while removing the interference effect from the conductors that carry the processor signals to the tiles. The pads shown in FIG. 2A refer to areas of metal that are gold wire bonded to the connecting terminals on a carrier for the device (not shown). Pads are shown only along 2 sides (rather than all four sides) of the array, in order that the substrate surface may be more easily be used for the growth of neuronal clusters.

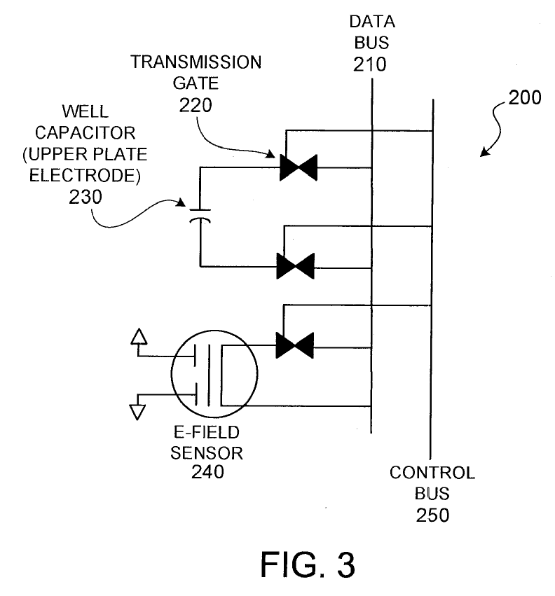

The array of tiles 110 comprises a 2-dimensional replication of a tile 140. Each tile 140 is configured to stimulate neurons in contact with it. Each tile 140 is independently electrically addressable. Structural details of the associated circuitry and connections are shown in the circuit schematic of FIG. 3, as discussed further hereinbelow. The number of distinct tiles can be controlled by the user by combining an adjacent group of tiles into one larger tile. This allows the stimulating electrodes on each tile to be connected together (thus reducing the processor overhead), and the transistor electric field sensor outputs to be combined to provide a stronger signal while also reducing processor overhead. By monitoring the position of neurons on the chip, adjacent groups of tiles may also be configured into shapes approximating those of the neurons.

The tiles are shown in FIG. 2A in a rectangular array, but the invention is not limited to such a configuration. For example, the array could be square in shape, such as a 2,000×2,000 tile square, or could be rectangular with some aspect ratio smaller or larger than the aspect ratio of the array shown in FIG. 2A. The array could still also be irregular in shape, such as a rectangular array having a corner cut-out, or could have the shape of a polygon, a circle, or an ellipse, or could be still other shapes. The array itself may occupy an area equivalent to, e.g., one square millimeter. It may also be equivalent to a square, 2 mm×2 mm, for example.

Preferably the tiles are dimensioned so that they can be placed on a sufficiently dense grid such that the spatial sampling of the electric field sensors will allow non-aliased capturing of the temporal/spatial changes of action potentials measured from the communicating neuronal network. Thus, in particular, the spatial sampling is designed to have an over-sampling: for example, it is preferable to sample spatially every 8 microns in order to capture full signal from action potentials. By contrast, e.g., sampling every 20 μ, or 50 μ, will not capture signal appropriately. The dimensions of each tile are preferably up to 10 μ or less on each side. A spacing of 8 microns, center-to-center, between tiles has been used in reported electric field sensor arrays (cf. the Infineon chip, cf. Eversmann, et al., “CMOS Sensor Array for Electrical Imaging of Neuronal Activity”, IEEE, 2005) The tiles also need not be square in shape, but can be triangular, rectangular, rhombohedral, or circular, or can be still other shapes such as other regular or irregular polygons.

Each tile further comprises a capacitor having an upper plate 150 and a lower plate 170. The upper plate is the upper metal layer electrode and the lower plate corresponds to the plate at the bottom of the well. Both upper plate 150 and lower plate 170 are, respectively, metal electrodes, Together, the capacitors associated with the array of tiles constitute an array of capacitors.

Preferably, the capacitor upper plate is fashioned from an upper layer metal in a standard CMOS process. Other metal or polysilicon layers may also be used, but they are less-preferred because they suffer from a reduced electric field voltage signal since they are situated further below the surface of the chip. The upper plate (electrode) may also be built by post-processing the surface of a standard CMOS process to provide, for example, electrodes on top of special high dielectric strength oxides (cf. the previously-referenced Infineon chip).

Each of the capacitor upper plates is configured so as to create an electric field at the surface of the substrate. By application of different voltages to different tiles, the electric field can be made to be non-uniform across the surface of the substrate.

The capacitor is further configured so as to provide a charge transfer stimulus to the neurons closest to it on the surface of the substrate, thereby allowing the microcontroller to communicate, via the tiles, with individual neurons on the surface.

In a preferred embodiment, an eletric field sensor is associated with each tile in order to detect neuronal electrical activity, such as signaling among the neurons, on the substrate. Electric field sensor 160 may be fabricated within the substrate. In the configuration shown in FIG. 2B, an electric field sensor is situated inside and in the same plane as a rectangular ring comprising the upper plate of the capacitor. Together, the electric field sensors associated with the array of tiles constitute an array of sensitive electric field sensors. As would be clear to one of ordinary skill in the art, the precise configuration of the upper and lower capacitor plates, and the field sensor, as shown in FIG. 2, is not limiting. Other possible configurations for a given tile include a U-shaped upper electrode enclosing the field sensor, or a square-shaped upper electrode that does not have an opening in the center. In the latter case, the electric field sensor lies adjacent to one side of the upper electrode, in the plane of the surface of the substrate.

The surface of the substrate preferably comprises a first area 120 having a culture growth area, and a second area (shown in FIG. 2A as covered with an array of tiles 110), the first area being adjacent to the second area. Other configurations of first and second area than that shown in FIG. 2A are permissible, such as one in which the first area wraps around more than one side of the array of tiles. Preferably, the first area comprises a culture growth area, is outside the area of the electrode array, and is at the periphery of the substrate.

The neurons are cultured in the first area that includes a medium containing growth permissive substances. The growth permissive substances preferably include substrate adhesion molecules, such as one or more of fibronectin, laminin and collagen. The electrode array generates an electrical field above the surface of the substrate in a controlled manner. Initial growth is guided by the electrode voltage waveforrns, which create an electrical gradient above the electrode array, thereby promoting growth along the direction of the electric field.

In some embodiments, one, or more agents such as trophic factors can be delivered directly using, for example, particles such as micro or nano-particles. In one embodiment, the capacitor structure formed on the substrate, when charged, generates an electric field, which will, in turn, release (“uncage”) trophic factors coated on nanoparticles such as quantum dots. The electric field can also be controlled to provide a lateral electrophoretic, or dielectrophoretic, force to focus the trophic factors around the growth cone of the growing neuron cell. In another embodiment, trophic factors can be introduced on to the substrate, and manipulated by electric fields and/or other means such as flow vectors introduced by micro-pumps. The trophic factors can be manipulated to provide a concentration gradient increasing from the growth cone(s) towards a target, for example, a neuron.

Each tile preferably has a well etched into it. Such a well may be etched directly into the substrate. In the embodiment shown in FIG. 2B, the bottom of the well is formed by the lower plate of the capacitor and the upper plate of the capacitor is at the top of the well. As shown in FIG. 2B, the upper plate partially overlaps the lower plate when viewed along an axis perpendicular to the surface of the substrate.

The dielectric between the lower and upper plates is, comprised of a medium in the well that contains trophic factors and/or nanoparticles. The nanoparticles are preferably quantum dots and, when quantum dots are used, they can be coated with gene perturbation molecules, such as double stranded RNA (RNAi) molecules. Such a configuration enables delivery of gene perturbation molecules to knock down the expression of specific genes in the neurons.

The quantum dots may also be labeled with fluorescent probes (such as rhodamine, etc.). The quantum dots can also be coated with trophic factors, for example, nerve growth factor (NGF), and epidermal growth factor (EGF).

In a preferred embodiment, the core of a QD is surrounded by a shell which is coated with polymer and streptavidin, which is conjugated with biotin to which EGF is further conjugated. These existing QD's can be selectively localized at a well housing in the electrodes on the substrate (this can be done either through micro-machining or trapping them with capacitive charge applied via the substrate, prior to cell plating. These QD's can then be “uncaged”. For example, in a preferred embodiment, a number of QD's are encapsulated into one core shell—such as a caged calcium compound, e.g., Nitro-5, that has calcium ions caged by a ligand-ring that releases the ions upon application of UV-light—and when desired, the shell can be broken by flashing intense UV light through the microscope. This approach is generally called flash photolysis. It is used to controllably release caged species such as calcium to reveal its biological actions within a cell. The QD's may also be controllably released by switching the charge polarity. For example, the core of the QD can be made to be either positively or negatively charged, and by applying either a negative or positive voltage they can be held at the capacitor site. When needed, the voltage can be switched to be of the same polarity as that of the core charge on the QD, thereby repelling the quantum dots from the holding site.

In some cases, the microcontroller can be used to control an integrated pump (e.g., a micro-pump) designed to deliver one or more agents (e.g., trophic factors or growth factors) to the surface of the substrate in, for example, a highly controlled and systematic manner. Such agents can be used to form a concentration gradient across the substrate surface. The electric field can be generated across the surface of the substrate, and, together, both the applied electrical field and the release of one or more agents (e.g., trophic factors) can be used to promote guided growth of a neuron. Such fields facilitate controlled growth of axons/dendrites in the neuronal culture and allow the manipulation of growth enhancing and/or marker molecules using techniques such as electrophoresis or dielectrophoresis. Dielectrophoresis is the motion of neutral, but polarizable, micro-particles, such as biological cells, in a nonuniform electric field. Dielectrophoresis results from the interaction between the field-induced polarization of the particle and the externally applied field. A constant phase non-uniform electric field causes particle conveyance either towards the electric field maxima (positive DEP) or minima (negative DEP), depending on the polarization state of the particle. A linear, phase varying, non-uniform electric field causes linear particle conveyance and particle rotation in the direction of movement known as traveling wave DEP.

The patterns of quantum dots labeled with various different biological molecules will enable selective labeling of a neuron to determine the role of the molecules. Moreover, once the quantum dots that are coated with trophic factors (such as NGF, EGF) are in contact with the growth cone, they will enable further growth promotion and neuronal survival over an extended time period. Concurrently, the quantum dot containing the fluorescent label is picked up by the growth cone and is endocytosed to be transported to the cell somata. This enables the effective fluorescent tagging of the neurons with a stable marker which can be imaged in live cells thus defining either the pre- or the postsynaptic identification of the cells and hence their synaptic sites. This provides a very powerful tool for neuronal imaging without having to compromise the viability of the cell through intracellular penetrations. All of these factors work together to promote well controlled and highly defined growth of neurons in cell culture.

Each tile preferably further comprises at least one transistor associated with it. Such a transistor is preferably a field effect transistor and can perform the role of a switch or a field-sensor, or one of each can be present. Together, the transistors associated with the array of tiles constitute a transistor switch array and are preferably individually addressable under the control of the microcontroller, which is connected to the tiles via conducting networks fabricated from metal and polysilicon layers on the chip that are below a metal ground plane that shields the electric fields, generated by these networks, from the chip surface where they could interfere with the electric fields generated by the array of tiles. The transistors may be further electrically connected to the plates of a two-dimensional array of capacitors.

Semiconductor technology is defined by a minimum width of standard conductor: the smaller this dimension, the more advanced is the level of technology. For use with the instant invention, the technology is preferably a 0.18 micron technology, or equivalent. Such a technology is suitable for constructing tiles on a 8 micron pitch because, for example, it can build a transistor within ˜1 micron. By contrast, the Infineon chip, referenced above, utilizes a 0.5 micron minimum conductor spacing, which would probably not be sufficient for the present application. Although smaller dimensions are generally more advantageous in other applications, as the applied voltages drop at a given location, they cannot support the E-fields required to stimulate neurons. Thus, 0.18 micron technology represents an acceptable compromise.

The microcontroller is preferably configured to accept programmable instructions to apply voltages to the tile array, through analog switches (transmission gates—see FIG. 3) contained within the tiles, and can therefore be used to generate arbitrary electric fields above the tiles.

The microcontroller is preferably also configured to accept programmable instructions to release growth-enhancing and/or marker molecules which can be controlled via electric fields in the dielectric (well) of the capacitor array. For example, the electrodes generating an electric field can be embedded in small cavities (such as wells) that contain charged (e.g., positively or negatively charged) quantum dots coated with one or more trophic factors. The quantum dots and their associated trophic factors can then be activated via a capacitive current applied through the electrode. The trophic factors and electric fields can cooperate to promote nerve growth.

The microcontroller controls the transistors and, preferably, the microcontroller is integrated with the transistors into an integrated circuit on the surface of the substrate. Not shown in FIG. 2A is a power source (optionally external) and/or an external computer, such as a host computer, that is configured to communicate with the integrated circuit(s). In this way, the microcontroller receives commands from the external computer. The computer may be a PC, PDA, laptop, notebook, or other suitable computer, and preferably comprises a user interface that permits a user to enter instructions and view results of measurements from the sensors. The power source can comprise, for example, a battery, or a source of mains voltage.

In FIG. 3, a capacitor 230 of a tile (not shown) has both of its plates connected to the data bus 210. The data bus carries analog signals whereas the control bus 250 carries digital signals to the transmission gates 220 which gate the appropriate analog signals onto the data bus. The control and data buses are connected to a processor (not shown) in the microcontroller, and to signal conditioning circuitry to enable reading of arbitrarily selectable sensors, and outputting of voltages onto arbitrarily selectable capacitor plates (electrode and/or lower well plate). The electric field sensor 240 is a transistor circuit (one or more transistors in appropriate configurations) that can have single or differential electric field sensing inputs (tile electrodes) and single or differential outputs connected to data bus 210. The advantage of a differential configuration (for example a differential pair) is that one of the input electrodes can be connected to a tile that is not below a neuron or its dendrite/axon and can be used to collect common mode signals from the conducting fluid which can be subtracted from the signals collected from the other electrode. Common mode noise is noise that is always present in the system; the goal here is to could subtract it out, without losing the signal, because the noise should be common to both measurements. A differential permits this subtraction and thereby increases signal-to-noise ratio. In this way common mode electric fields (e.g., noise) can be reduced. One of ordinary skill in the art would understand that there are many different circuit structures available to interconnect the tiles via the data and control buses (e.g., linear or matrix connection) depending on the preferred embodiment of the array.

The microcontroller preferably comprises a power source, for powering the integrated circuits, and may further be connected to a host computer, such as a microcomputer, through either a dedicated link, such as ethernet, firewire, or USB connection, or via a wireless connection. The microcontroller can further comprise software and firmware that controls the selection of tiles to which voltages are applied.

In some embodiments, the device comprises a wired or wireless connection to the external microcomputer and power source. The external microcomputer controls the activation of the electric field. The power source provides the power for the controller and tile array used to generate the electric field and control the trophic factor release (via uncaging, micropump fluid flow, and the like).

In some embodiments, a device provided herein includes functionality that allows the sensing of the progression of nerve fibers across the surface. In one embodiment, the progression can be monitored using impedance measuring means. As the neuron cells progress across the surface, the applied field and delivery of trophic factors are controlled by the two-dimensional array of tiles such that the field and trophic factors are limited to regions ahead of the neuron cells. Thus, when the fibers reach a specific tile, the next adjacent tile ahead of the neurons can be activated, which results in the application of an electric field, and the release of trophic molecules beyond the ends of the axon. The array of electrodes adjacent to the growth cone of the neuron can intermittently stimulate the neuron through electric field stimulation. This process continues as the neurons grow across the substrate. These alternating on and off responses in various capacitors and electrodes within the device cause progressive nerve growth.

One advantage of the apparatus and method of the present invention is that, because the neurons grow in a guided direction, a neuronal network can be observed within a short period of time, such as a week. By contrast, undirected culturing of cells on a surface, in which, for example, trophic factors are to be found uniformly everywhere, leads to build-up of neuronal networks in typically as long as 8-10 weeks.

A further advantage of the present invention is that the neuronal networks that are grown more closely resemble structures seen in an intact brain than to neuronal networks grown in environments where trophic factors are deposited uniformly. For example, typically in neuronal cultures, cells grow out on all sides and it is difficult to distinguish between the axon and dendrites from a given cell; in a real brain, the neurons have a restricted form, with one extended axon, and multiple dendrites.

The details of one or more embodiments of the invention are set forth in the accompanying drawings and the description below. Other features, objects, and advantages of the invention will be apparent from the description and drawings, and from the claims.

### EXAMPLES

### Example 1

### Electrode Array and Microcontroller

The following describes an electrode array which, although it does not provide an array of capacitors, embodies various design principles that are consistent with the apparatus of the present invention. The design of an electrode structure (lexel array) for a bio-analysis system is a checkerboard pattern of discrete planar metal microelectrodes. The lexel array has been fabricated using the services of CMC Microsystems (formerly the Canadian Microelectronics Corporation) using the TSMC 0.18 μm mixed signal CMOS process with 3.3 V devices. The checkerboard pattern allows for the maximum electrode density per unit area for the chosen circuit topology and the available fabrication process. The distance across the lexels and the spacing between the lexels is 10 μm.

The designed array contains 741 lexels, constrained only by the available silicon area. In order to keep the number of control signals manageable while still having the ability to impose arbitrary voltages (potentials) to each individual electrode, a conventional sample and hold circuit comprised of a transmission gate and capacitor is implemented at each lexel, where the lexel is the top plate of the capacitor. A ground plane covers the spaces between lexels to shield the particles from the stray fields of the active devices and routing conductors.

The lexel circuitry is arranged into rows and columns. Again, to manage the number of control signals for this prototype, a demultiplexer is used between adjacent rows to reduce the number of required row signals by a factor of two. This arrangement also halves the number of column signals required. The layout of the lexelsis in a checkerboard fashion, and each lexel comprises a hexagonal upper metal electrode.

When a particular row is selected the transmission gate for each lexel in the row is activated. The input signal for each column is then sampled by the corresponding lexel in the activated row. Each column uses a basic time division multiplexing scheme where each lexel signal is allocated a particular time slot in a rotation. By continuously updating the signal levels in each row, an arbitrary electric field can be synthesized. Rows can be simultaneously activated so that the symmetry that exists in many of the electric field shapes used in dielectrophoretic manipulation of cells (the main target of the lexel array) can be exploited.

The lexel array is programmed using the Xilinx XCV2000E FPGA board from an ARM Integrator/AP Rapid Prototyping Platform (RPP) provided by CMC Microsystems. A custom printed circuit board has been fabricated to interface the lexel array microchip to the FPGA board. The custom PCB uses an Analog Devices AD9748 digital-to-analog converter along with an Analog Devices AD8041 operational amplifier to provide the analog signals, required to drive each column of lexels. A look up table is used to multiplex the required signals, and a number of look tip tables are stored on the FPGA. The FPGA also controls the transmission gates so that the proper signal is sampled at each lexel. The dynamic assignment of the appropriate look up table for each column enables the programmable and reconfigurable capabilities of the electric field that is generated.

### Example 2

### Capacitative Stimulation of a Synapse

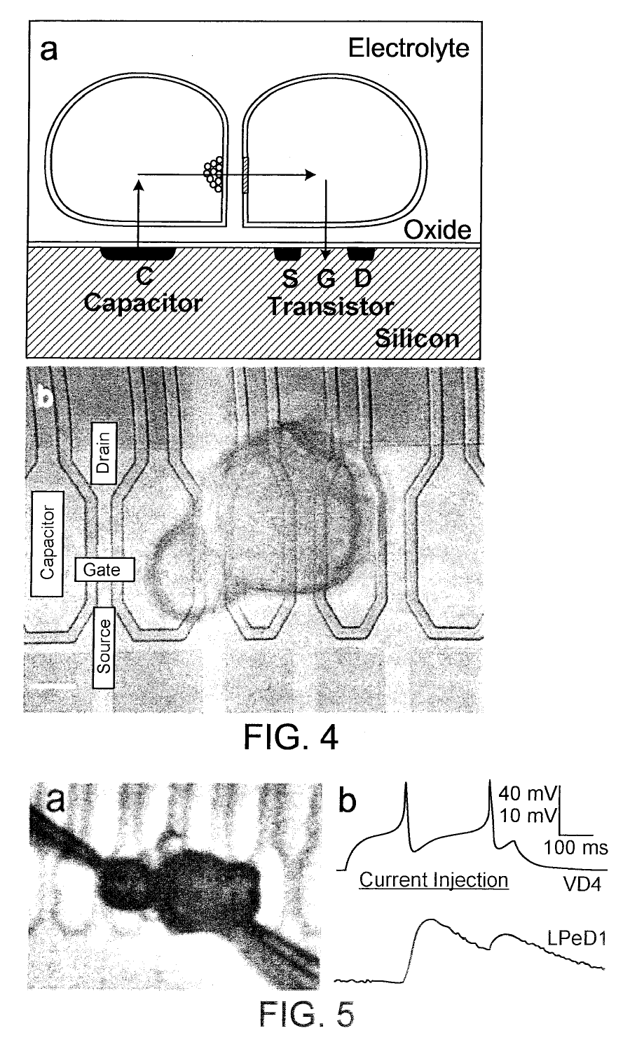

FIG. 4 shows a silicon chip interfaced with a synapse (not to scale; scale bar 20 μm.): (a) Hybrid device with capacitor (C), chemical synapse, and transistor (gate G, source S, drain D); (b) Micrograph with presynaptic VD4 neuron (left) and postsynaptic LPeD1 neuron (right) from Lymnaea stagnalis on a linear array of capacitors and transistors. The implementation of a neuronal memory on a semiconductor required a microelectronic interfacing of two neurons that formed a chemical synapse as illustrated in FIG. 4(a). It is known that in vivo the neuron VD4 from L. stagnalis forms a cholinergic synapse with the neuron LPeD1. That synapse can be reconstituted in vitro in a soma-soma configuration. By using soma-soma contacts, problems with a displacement of neurons from their contact sites as caused by neuronal outgrowth were avoided. VD4 (visceral dorsal 4) and LPeD1 (left pedal dorsal 1) neurons were paired on a linear array of upon the synaptic connectivity between many neurons microelectronic contacts for stimulation and recording as within a network and their ability to exhibit synaptic illustrated in FIG. 4(b). Presynaptic action potentials were elicited by a capacitor; pre- and post-synaptic activities were recorded by transistors. Capacitor stimulation was applied to potentiate synaptic strength.

Transistors and capacitors were made by boron doping of n-type silicon. They were insulated from the electrolyte by a 10 nm layer of silicon dioxide and from one another by narrow lanes of 600 nm local field oxide. The chips were wire bonded to a standard package (Spectrum, CPGA 208L, San Jose, Calif., USA). A Perspex chamber was attached for the culture medium. Before each use, the chip was wiped with a 10% solution of detergent (FOR, Dr. Schnell, Munich, Germany) in milli-Q water of 70° C., rinsed with milli-Q water, and sterilized with UV light for 15 min. A solution of poly-L-lysine (MW 84,000, P-1274, Sigma, Munich, Germany) was applied for 8 h (1 mg=ml in 150 mM tris, pH 8.4). Finally the chip was rinsed three times with aqua ad (Braun, Melsungen, Germany), once with antibiotic saline, and three times with aqua ad and dried. To obtain individual VD4 and LPeD1 neurons of L. stagnalis, the central ring of ganglia of snails (shell length 15-22 mm) was isolated and the neuronal somata were extracted with a suction pipette (60-90 μm diameter) attached to a syringe (Gilmont GS-1200 2 ml, VWR, Brisbane, Calif., USA) by a polyethylene tubing. Pairs of VD4 and LPeD1 neurons were incubated for 12-18 h on the chip in 1 ml conditioned medium at room temperature and 80% humidity.

FIG. 5 shows neurons with soma-soma synapse on a silicon chip. FIG. 5(a) shows a micrograph of three neurons on a silicon chip with the central LPeD1 and the left VD4 impaled by micropipette electrodes. FIG. 5(b) shows intracellular recording: upper trace: two action potentials in VD4 elicited by a current injection of 1 nA (holding voltage—60 mV); lower trace: excitatory postsynaptic potentials (EPSPs) in LPeD1 (holding voltage VD4—90 mV). FIG. 5(a) shows a chip with the pair of VD4 and LPeD1 neurons that was used. First, whether a chemical synapse was formed was tested for. Conventional intracellular recordings were made from the pre- and post-synaptic neurons. The cells were impaled with microelectrodes made from borosilicate capillaries (1403547, Hilgenberg, Malsfeld, Germany) with a puller (Zeitz, Augsburg, Germany), filled with a saturated solution of K₂SO₄ (15-20 MΩ), contacted with chlorinated silver wires and connected to bridge amplifiers (BA-1S, NPI Instruments, Tamm, Germany). Spontaneous spiking was suppressed by a hyperpolarizing current. Action potentials were elicited by depolarizing pulses of 0.3 nA and 500 ms. Presynaptic action potentials in VD4 generated 1:1 excitatory postsynaptic potentials (EPSPs) in LPeD1, as shown in FIG. 5(b) (n=6). These were similar to those seen in vivo and in vitro.

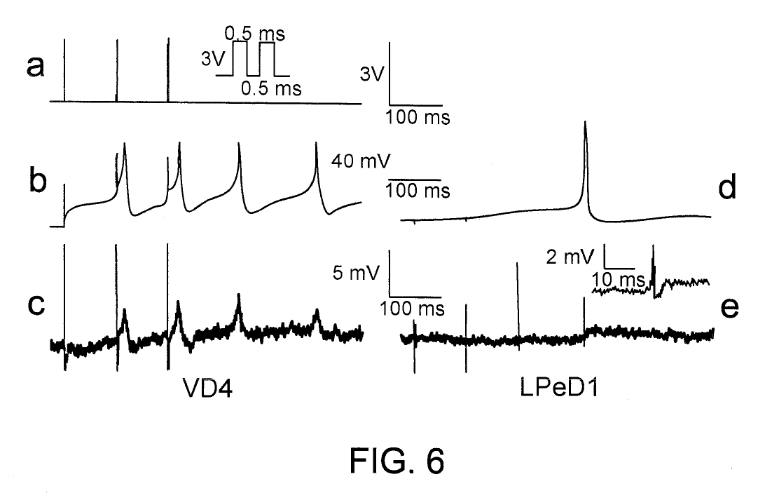

FIG. 6 shows synaptic transmission on silicon chip. FIG. 6(a): voltage at the capacitor beneath VD4 with three double-pulse stimuli (blowup). FIG. 6(b): shows intracellular voltage of VD4 with four action potentials (holding voltage ˜60 mV). FIG. 6(c): transistor record of VD4 with responses to the presynaptic action potentials. FIG. 6(d): intracellular voltage of LPeD1 with one postsynaptic action potential (holding voltage—70 mV). FIG. 6(e): transistor record of LPeD1 with the response to the postsynaptic action potential (blow-up). The short transients in the transistor records are due to extracellular voltages beneath the neuron pair and to electrical cross talk on the chip.

Whether action potentials in VD4 could be elicited from the chip by capacitor stimulation was tested for, while keeping the cell impaled with a microelectrode. Using a waveform generator (33120A, Hewlett-Packard, Palo Alto, Calif., USA) positive voltage pulses were applied to a capacitor (bulk silicon at +7.5 V) with respect to the bath at ground potential (Ag/AgCl electrode). A stimulus consisted of two pulses with +3 V amplitude and 0.5 ms (FIG. 6(a)). A sequence of three paired pulses gave rise to short responses of the intracellular voltage that induced sustained intracellular depolarizations (FIG. 6(b)). Action potentials generally occurred after the second or third stimulus.

Excitatory postsynaptic potentials in LPeD1 were induced by capacitively elicited activity in VD4, as observed by intracellular recording (not shown). The EPSPs were indistinguishable from EPSPs induced by intracellular stimulation of VD4. Yet, we focused on the presynaptic stimulation of postsynaptic action potentials, because transistor recording of EPSPs was not possible due to noise. In fact, with intracellular recording we observed postsynaptic action potentials after two to three presynaptic spikes elicited by capacitor stimulation (FIG. 6(d)), in analogy to intracellular presynaptic stimulation. The result provides experimental evidence that capacitor stimulation is able to trigger synaptic transmission (n=4).

To complete the interfacing of synaptic transmission by the silicon chip, whether pre- and post-synaptic activity could be observed with transistors was tested for. Before each measurement, the transistors (source at +2.5 V, drains at +0.5 V, source-drain current 50-100 μA) were calibrated by applying defined voltages to the bath. Voltage pulses were applied to a capacitor beneath a VD4 neuron. Short transients appeared in both transistor records beneath VD4 and LPeD1 (FIGS. 6(c) and 6(d)) due to extra-cellular voltages beneath the neuron pair and to electrical cross talk on the chip. The action potential in the presynaptic VD4 neuron was recorded by a transistor as a positive transient of extracellular voltage with an amplitude around 3 mV (FIG. 6(c)). The action potential elicited in the LPeD1 neuron by synaptic transmission was recorded by a transistor as a sharp peak of about 3 mV in its rising phase (FIG. 6(e)). This experiment demonstrates the interfacing of a chemical synapse by a semiconductor chip with presynaptic capacitor stimulation and pre- and postsynaptic transistor recording. The results with intra-cellular monitoring of both cells (n=3) were confirmed by experiments without impaling LPeD1 (n=2) and without impaling either cell (n=2) (not shown).

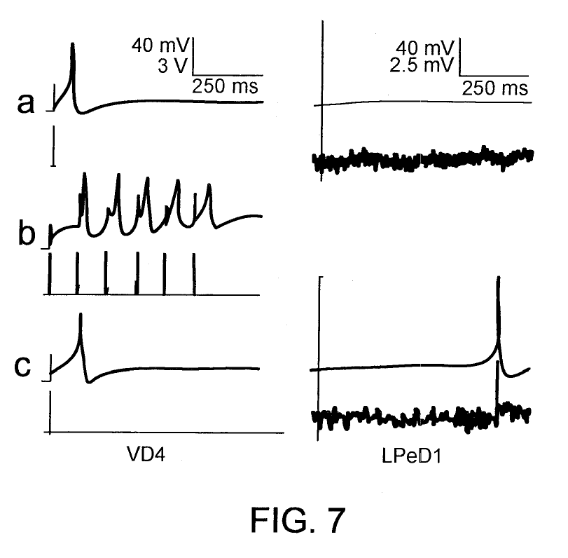

A particularly interesting aspect of the VD4-LPeD1 synapse is its capability to exhibit short-term potentiation that is thought to form the basis of working memory in animals. Specifically, a presynaptic tetanus in VD4 consisting of five to ten action potentials enhances the amplitude of subsequent EPSPs which generate post-synaptic spikes in LPeD1. FIG. 7 shows synaptic potentiation on a silicon chip. The upper traces show intracellular voltages, the lower traces capacitor stimuli (left) and transistor records (right). FIG. 7(a): Control; capacitor stimulation of VD4 neuron with action potential in VD4 (left) and no postsynaptic action potential in LPeD1 (right). FIG. 7(b): Potentiating stimulus; train of six capacitor stimuli applied to VD4 with action potentials. FIG. 7(c): potentiated response. Capacitor stimulation of VD4 with action potential in VD4 (left) and postsynaptic action potential in LPeD1 (right).

At first, short-term plasticity on the chip with current injection through intracellular electrodes (n=5, not shown) was demonstrated. Then, whether the potentiation could be elicited by capacitor stimulation and recorded with a transistor was tested. Since transistor recording of EPSP's was not possible, the potentiation was probed by the appearance of postsynaptic action potentials. First as a control, a single action potential was elicited in VD4 by a pair of voltage pulses applied to the capacitor. In that case postsynaptic depolarization was not sufficient to elicit an action potential in LPeD1 (FIG. 7(a)). Then a capacitive tetanus of six single voltage pulses (3 V, 0.5 ms) was applied that triggered five action potentials in VD4 (FIG. 7(b)). To test for potentiation, again an action potential was elicited in VD4 a few seconds after the tetanus (FIG. 7(c)). The post-tetanic action potential in the presynaptic cell reproducibly caused a postsynaptic spike in LPeD1 that was recorded by the transistor (FIG. 7(c)). The experiment shows that the modulation of a soma-soma synapse can be directly induced and monitored by the silicon chip (n=2).

The foregoing description is intended to illustrate various aspects of the present invention. It is not intended that the examples presented herein limit the scope of the present invention. The invention now being fully described, it will be apparent to one of ordinary skill in the art that many changes and modifications can be made thereto without departing from the spirit or scope of the appended claims.

What is claimed:

1. A system for guiding growth of neurons, the system comprising:
an array of tiles on a surface;
a medium contacting the surface, wherein the medium contains:
one or more neurons; and
growth permissive substances; and
a microcontroller electrically connected to the array of tiles, and configured to cause an electrical voltage to be applied selectively to at least one of the tiles, thereby causing one or more neurons in contact with the at least one tile to grow.

2. The system of claim 1, wherein the surface comprises a first area and a second area, said first area being adjacent to said second area.

3. The system of claim 2, wherein the first area contains the array of tiles and the neurons are guided to the second area.

4. The system of claim 1, further comprising a transistor associated with each tile.

5. The system of claim 4, wherein the transistors constitute a transistor switch array and are individually addressable.

6. The system of claim 5 wherein the microcontroller controls the transistors.

7. The system of claim 6 wherein the microcontroller is integrated with the transistors into an integrated circuit.

8. The system of claim 1, wherein the surface is a silicon die.

9. The system of claim 1, wherein each tile comprises a capacitor having an upper plate and a lower plate.

10. The system of claim 9 wherein the capacitor upper plate is fashioned from upper layer metal of a CMOS process.

11. The system of claim 9 wherein the capacitor is configured so as to provide a charge transfer stimulus to the neurons, thereby allowing the microcontroller to communicate with individual neurons on the surface.

12. The system of claim 9 wherein each of the capacitor upper plates are configured so as to create an electric field on the surface.

13. The system of claim 12 wherein the electric field is non-uniform.

14. The system of claim 9 wherein an electric field sensor is fabricated within the tile in order to detect signaling among the neurons.

15. The system of claim 9 wherein each tile has a well etched into it.

16. The system of claim 15 wherein the lower plate of the capacitor forms the bottom of the well.

17. The system of claim 15 wherein the upper plate of the capacitor is at the top of the well and partially overlaps the lower plate when viewed along an axis perpendicular to the surface.

18. The system of claim 15 wherein the well contains growth enhancing molecules or nanoparticles.

19. The system of claim 1, wherein the microcontroller is connected to a computer.

20. The system of claim 19, wherein the microcontroller is connected to the computer via a wireless connection.

21. The system of claim 1, wherein the growth permissive substances include substrate adhesion molecules.

22. The system of claim 21, wherein the substrate adhesion molecules include at least one of fibronectin, laminin and collagen.

23. The system of claim 1, wherein the growth permissive substances include Schwann cells.

24. The system of claim 1, further comprising one or more growth-enhancing molecules.

25. The system of claim 1, wherein the growth enhancing molecules include trophic factors.

26. The system of claim 25, wherein the trophic factors include at least one of nerve growth factor (NGF) and epidermal growth factor (EGF).

27. The system of claim 1, further comprising nanoparticles, wherein the nanoparticles are arranged in a pattern on the surface.

28. The system of claim 27, wherein the nanoparticles are quantum dots.

29. The system of claim 28, wherein the quantum dots are coated with gene perturbation molecules.

30. The system of claim 29 wherein the gene perturbation molecules are double stranded RNA molecules.

31. The system of claim 28, wherein the quantum dots are coated with trophic factors.

32. The system of claim 31, wherein the trophic factors include at least one of nerve growth factor (NGF) and epidermal growth factor (EGF).

33. The system of claim 1, wherein each tile in the array of tiles is independently electrically addressable.

34. A method for guiding growth of neurons, the method comprising:
culturing neurons on a substrate surface;
patterning growth permissive substances, trophic factors, and nano particles on the substrate surface;
fabricating an array of tiles on the surface; and
applying an electrical voltage to at least one of the tiles, thereby stimulating growth in a neuron in contact with the at least one tile.

35. The method of claim 32, wherein each of the tiles in the array of tiles is independently electrically addressable.
